(19) United States
(12) Patent Application Publication
(10) Pub. No.: US 2007/0073086 A1
(43) Pub. Date: Mar. 29, 2007

(54) Process for manufacturing diphenylamines

(75) Inventor: Ponnampalam Mathiaparanam, Appleton, WI (US)

Correspondence Address: APPLETON PAPERS INC.;LAW DEPARTMENT, 825 E. WISCONSIN AVENUE, PO BOX 359, APPLETON, WI, 54912-0359, US

(73) Assignee: Appleton Papers Inc., Appleton, WI (US)

(21) Appl. No.: 11/236,539

(22) Filed: Sep. 28, 2005

(51) Int. Cl. C07C 209/02; C07D 207/04; C07D 265/30

(52) U.S. Cl. 564/433; 546/229; 548/577; 544/162

(57) ABSTRACT

The invention teaches novel process steps for the rapid high yield manufacture of diphenylamines of the formula 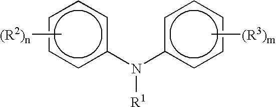
wherein R¹ is selected from hydrogen, alkyl, and aryl;
wherein each R² and R³ is the same or different and each is independently selected from hydrogen, alkyl, alkoxy, aralkyl, dialkylamino, alkylarylamino and substituted or unsubstituted aryl, the substituents on aryl being each independently selected from alkyl (C₁-C₈), alkoxy (C₁-C₈), aroxy, aralkoxy and halogen; wherein n and m are each independently an integer from 1 to 5. Diphenylamines are key intermediates for the production of leuco dyes used in pressure-sensitive and heat-sensitive imaging systems. The process in at least one embodiment comprises reacting at elevated temperature an aryl halide with an aromatic amine in an organic solvent and aqueous alkaline solution and optionally in some embodiments, phase-transfer agent, followed by addition of catalytic amounts of bis[tri(t-butylphosphine)]palladium at a suitable temperature to rapidly form diphenylamine.

### BACKGROUND OF THE INVENTION

1. Field of the Invention

This invention relates to processes of preparation of leuco dyes, and more particularly to processes for preparation of certain intermediates useful in manufacture of leuco dyes. The invention in particular teaches a novel process for manufacture of diphenylamines. Diphenylamines are useful intermediates in the preparation of leuco dyes. Leuco dyes find extensive application in pressure-sensitive and heat-sensitive imaging systems or record materials.

2. Description of the Related Art

This invention relates to a process for manufacturing diphenylamines of formula (1). Diphenylamines are key intermediates for the production of leuco dyes used in pressure-sensitive and heat-sensitive imaging systems.

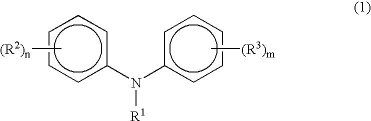

In formula (1), R¹ is selected from hydrogen, alkyl, and aryl; each R² is the same or different and each R³ is the same or different and are each independently selected from hydrogen, alkyl, alkoxy, aralkyl, dialkylamino, alkylarylamino, pyrrollidino, piperidino, morpholino and substituted or unsubstituted aryl, the substituents on aryl being each independently selected from alkyl (C₁-C₈), alkoxy (C₁-C₈), aroxy, aralkoxy and halogen; and n and m are each independently an integer from 1 to 5.

Leuco dyes used in pressure-sensitive and heat-sensitive imaging systems may contain either fluoran (2) or triphenylmethane (3) moieties.

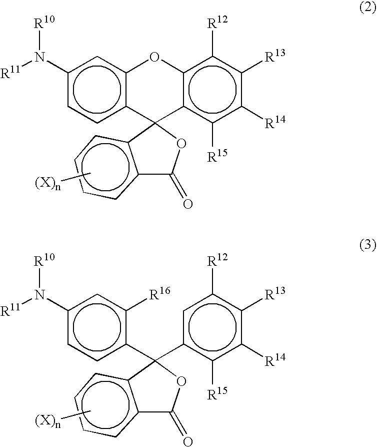

In formulas (2) and (3) R¹⁰ and R¹¹ are independently alkyl (C₁-C₈), aralkyl, or aryl, R¹⁰ and R¹¹ may also form alicyclic (pyrrollidine, piperidine or morpholine) rings with nitrogen; R¹² -R¹⁶ may be different or same and each independently represents hydrogen, alkyl (C₁-C₈), alkoxy, aralkyl, dialkylamino, alkylarylamino and substituted or unsubstituted aryl. X may be independently hydrogen, alkyl (C₁-C₄), dialkylamino or halogen and n is an integer 1 to 5.

Leuco dyes according to formulas (2) and (3) are prepared by condensing a keto acid with a diphenylamine in an acidic medium or in acid anhydrides such as acetic anhydride. For example:

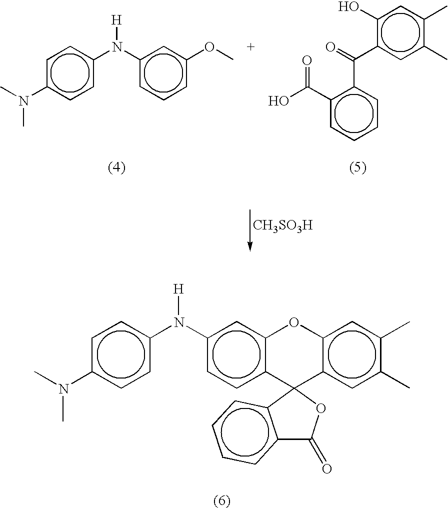

The keto acid may be prepared by reacting a diphenylamine with a substituted or unsubstituted phthalic anhydride. For example:

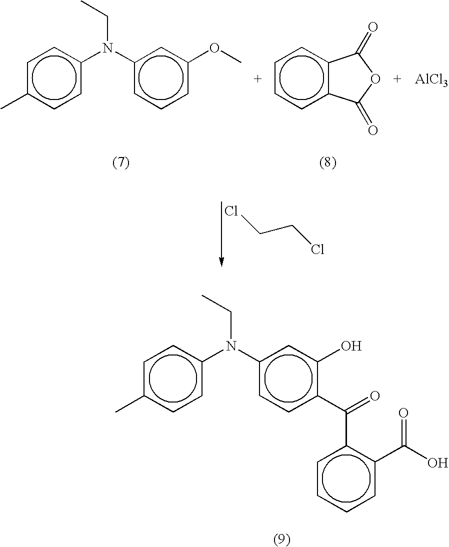

Furthermore, keto acid (9) can be condensed with another or different diphenylamine (10) to form a leuco dye (11) as shown below. In the production of leuco dye (11) two different diphenylamines are used. These examples illustrate the importance of diphenylamines in the production of leuco dyes.

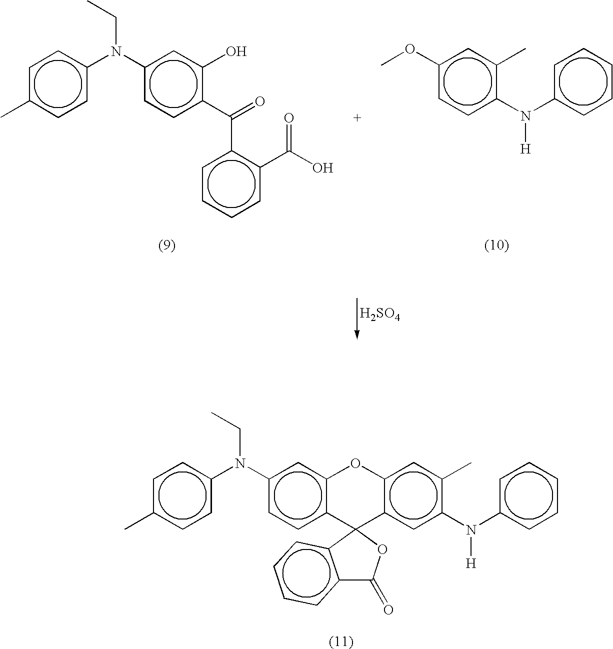

Diphenylamines are commercially important materials by virtue of their use as intermediates in the manufacture of various leuco dyes. Such dyes or chromogenic materials find applications in pressure-sensitive and heat-sensitive imaging systems and other indicator applications. Diphenylamines are typically prepared by the condensation of aromatic amines and resorcinol with the removal of water. These condensations require very high temperatures in an inert atmosphere for a considerable amount of time and the product is difficult to isolate because of tarry side products.

For example, Gnehm et al. teaches N,N-dimethyl-4-phenylenediamine (12) and resorcinol (13) are heated at 200° C. in an atmosphere of carbon dioxide to yield 4-dimethylamino-3′-hydroxydiphenylamine (14) [R. Gnehm and G. Weber, J. Prakt. Chem., 177, 223 (1904)].

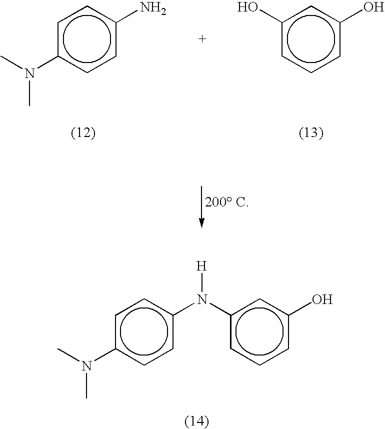

Another method for preparation of diphenylamines (19) uses a Goldberg reaction [I. Goldberg, Ber. Dtsch. Chem. Ges., 39, 1691 (1906); H. S. Freeman, J. R. Butler and L. D. Freeman, J. Org. Chem., 43, 4975 (1978)]. In a Golberg reaction, the aromatic amine (15) is converted by N-acetylation to acetanilide (16) which is heated with aryl halides (17), copper catalyst and potassium carbonate or potassium acetate at 175-250° C. for several hours to form N-acetyldiphenylamines (18). The N-acetyldiphenylamines are then hydrolyzed using either sodium hydroxide or potassium hydroxide in a suitable solvent. Although this is believed to be a presently practiced method to manufacture diphenylamines used in the production of leuco dyes for pressure-sensitive and heat-sensitive paper systems, the stringent reaction conditions such as high temperatures over long periods of time and difficult work up of dark crude products coupled with a three step sequence makes this method unattractive. That this method is used is a reflection of the limited known pathways to produce commercial quantities of diphenylamines.

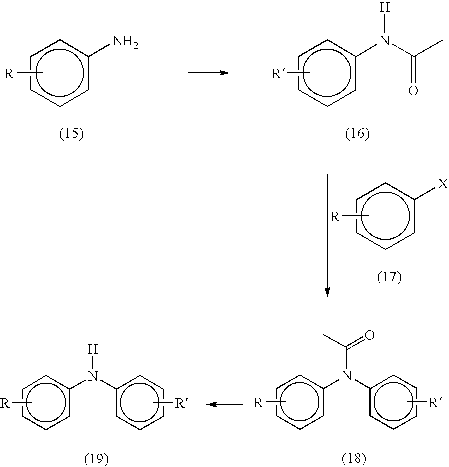

Diphenylamines (23) have been taught as able to be prepared by condensing aminophenols (21) with aromatic amines (20) using titanium (IV) isoproxide (22) in toluene [T. Obitsu, Y. Ohnishi, S. Yoshinaka, M. Koguchi, M. Yanagita and N. Hirai, U.S. Pat. No., 4,954,631, Sep. 4, (1990)]. The difficulties of handling extremely hygroscopic titanium (IV) isopropoxide on a larger scale however makes this method not amenable to commercial scale manufacture of diphenylamines.

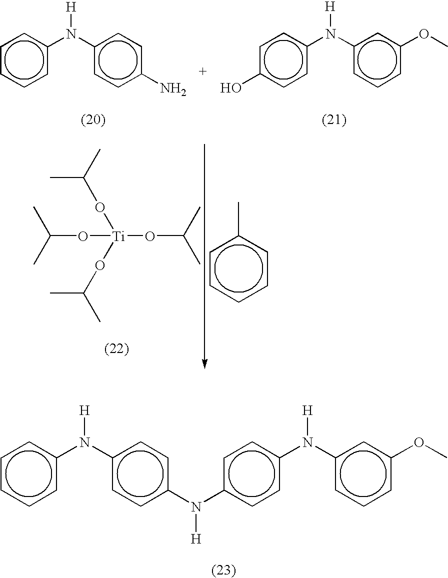

In 2002, Kuwano et al. taught amination of aryl halides (17) by blending inexpensive alkali hydroxides and bis[tri(t-butylphosphine)]Pd [0] (26) to form arylamines [R. Kuwano, M. Utsunomiya and J. Hartwig, J. Org.Chem., 67, 6479 (2002)]. “[0]” refers to valence state of palladium. This reaction was taught in toluene using cetyltrimethylammonium bromide (27) as a phase-transfer agent. Reported yields after three hours of reaction were poor with various listed phase transfer catalysts, and did not exceed 20%. Protracted reaction times of 24 hours improved yields of diarylamines (1) for which high yields were reported only with cetyltrimethylammonium bromide as a phase-transfer catalyst.

### DETAILED DESCRIPTION

The present invention is an improved process for manufacture of diphenylamines of the formula (1): 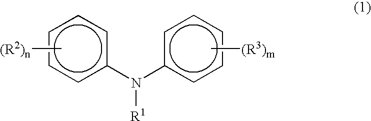
wherein R¹ is selected from hydrogen, alkyl, and aryl;
wherein R² and R³ are each independently selected from hydrogen, alkyl, alkoxy, aralkyl, dialkylamino, alkylarylamino, pyrrollidino, piperidino, morpholino and substituted or unsubstituted aryl, the substituents on aryl being each independently selected from alkyl (C₁-C₈), alkoxy (C₁-C8), aroxy, aralkoxy and halogen;
wherein n and m are each independently an integer from 1 to 5.

The process comprises reacting an aryl halide with an aromatic amine in an organic solvent and aqueous alkaline hydroxide and a phase transfer agent to which catalytic amounts of bis[tri(-butylphosphine]palladium are added at a suitable temperature.

The phase transfer agents can be selected from those generally known to the skilled artisan, and include by way of illustration and not limitation, crown ethers such as 1,4,7,10,13-pentaoxacyclopentadecane; 1,4,7,10,13,16-hexaoxacyclooctadecane (18-Crown-6); 1,4,7,10-tetraoxacyclododecane (12-Crown-4); dibenzo-18-crown-6-dibenzyl-24-crown-8; dicyclohexano-18-crown-6; dicyclohexano-24-crown-8; tetramethylammonium chloride; tricaprylmethylammonium halide; cetyltrimethylammonium halide such as cetyltrimethylammonium bromide; tetra-n-butylammonium halide; quartemary ammonium salts; quartemary ammonium phosphates; pyridinium salt, cetyl pyridinium bromide; and benzyldimethyltetradecylammonium chloride. The amount of the phase transfer agent is generally a catalytically effective amount and generally less than 10% by weight, preferably 1% or less by weight, and more preferably 0.5% or less.

More particularly, the process comprises addition of an arylamine of the formula (24) 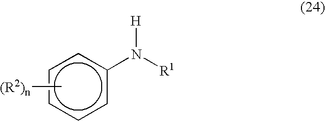
and an aryl halide of the formula (25) 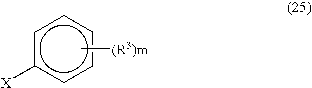
to a water immiscible solvent forming a mixture. An aqueous alkaline solution is added to the mixture and the mixture is agitated such as by stirring. The mixture can be a blend or emulsion for purposes of this process or optionally enough aqueous alkaline solution can be added to form a separate aqueous phase, though a visibly distinct separate phase is not required for the process.

The mixture is heated to equilibrate the system. Heating is continued to a temperature in excess of 40° C. and more preferably in excess of 80° C., most preferably 80° to 95° C.

After the mixture is brought to temperature, then a catalytically effective amount of a palladium catalyst of Pd [P(t-Bu)₃]₂ is added. Pd [P(t-Bu)₃]₂ is bis[tri(t-butylphosphine] palladium [0]. The amount of the palladium catalyst is less than 10% by weight of the mixture and preferably 1% or less by weight, and more preferably 0.5% or less.

Diphenylamine is rapidly formed by this process. Reaction speed can also be influenced by catalyst concentration, with slower reactions seen at lower catalyst concentration. Reaction speed of the process is generally less than four hours, and often a matter of minutes. The examples herein illustrate reaction times from 15 minutes to 2.5 hours involving refluxing the mixture for a time and temperature sufficient to form the diphenylamine.

The inventor has discovered that surprisingly diphenylamines can be made rapidly and in high yield while in one embodiment effectively using less expensive phase transfer agents. In an alternate embodiment, the phase transfer agent can be omitted.

The amination of aryl halides with arylamine can be made to proceed dramatically faster. Kuwano, for example, reports either poor yields in the attempted amination of p-chlorotoluene, or long reaction times of at least 24 hours.

By appropriate selection of reaction conditions and sequence of addition steps, the present invention surprisingly was able to produce diphenylamine in high yield in as little as fifteen minutes. With some phase transfer agents, the reaction time was at least 2.5 hours, but even this is about ten times faster or a magnitude of order faster than any previously described method.

In a preferred embodiment, the improved process of the invention involves the addition of arylamine (or arylamine can be generated in situ from arylamine salts) and aryl halide to a water immiscible solvent such as toluene or other hydrocarbon in a flask equipped with a mechanical stirrer and reflux condenser followed by the addition of 50% aqueous alkali and phase-transfer agent; heating the contents of the flask to a uniform 85-90° C. with vigorous stirring; and adding the catalyst last. The reaction time reduction while employing less expensive reagents and high yields of products are much sought after features in a manufacturing process.

Aryl bromides were found to react faster than aryl chlorides. For example, N-ethyl-4-toluidine[(24), R₂=4-methyl and R₁=ethyl] and 3-bromoanisole[(25), R₃=3-methoxy and X=Br] react under the above-mentioned conditions to give N-ethyl-3-methoxy-4′-methyldiphenylamine[(1), R₂=4′-methyl, R₃=3-methoxy and R₁=ethyl, n=1, m=1] within 15 minutes in almost quantitative yield. By contrast, the reaction of N-ethyl-4-toluidine with 3-chloroanisole[(25), R₃=3-methoxy and X=Cl] took 2.5 hours to completion to give the same diphenylamine in almost quantitative yield.

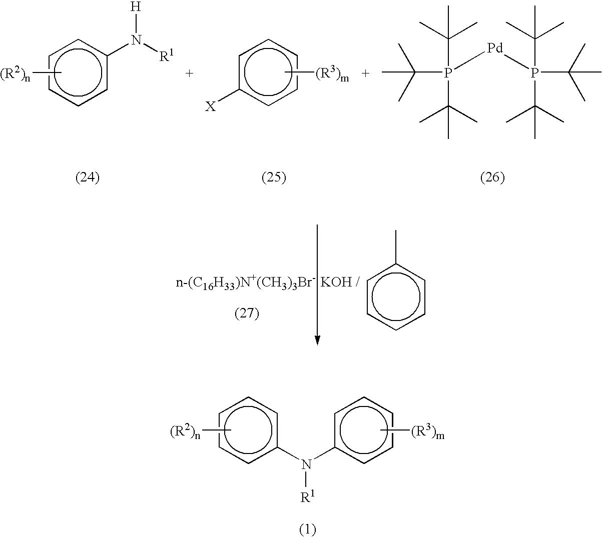

Phase-transfer agents such as cetyltrimethylammonium bromide, tricaprylmethylammonium chloride (aliquat 336) and tetra-n-butylammonium bromide (TBAB) catalyzed the reaction to completion giving almost quantitative yields. Quarternary ammonium salts with one or more long alkyl chains (12 carbons or more) are preferred. Quarternary phosphonium salts can also be substituted for quarternary ammonium salts. The catalysts can be used individually or, as blends. Individual catalysts were preferred. The temperature range in which the reaction proceeds is 40°-100° C., and the preferred range is 80-95° C.

Examples 5 and 7 herein illustrate the versatility of the process of the invention. One can start with an arylamine (24) and aryl halide (25), or alternatively an acid salt, (28) or (30), of the arylamine can be used as a starting material. The aqueous alkaline solution then also serves to generate the aromatic amine in situ.

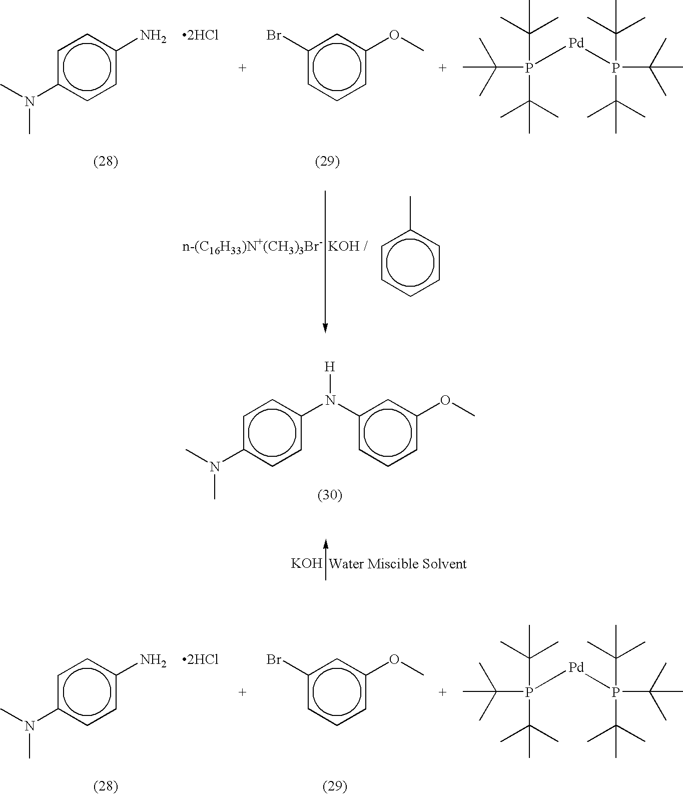

In a yet further alternative embodiment the arylamine (24) or the acid salt (30) of the arylamine together with the aryl halide (25) can be dissolved or dispersed in a water miscible solvent to form the mixture. The water miscible solvent can include water miscible solvent such as 1,4-dioxane, tetrahydrofuran, noncyclic or cyclic ethers, ethylene glycol dimethylether (glyme), diglyme, triglyme, and tetraglyme, acetonitrile, dimethylsulfoxide, dimethylformamide, monopropylether methyltertbutylether, and ethylene glycol monopropyl ether by way of illustration and not limitation. With use of the water miscible solvent, advantageously the phase transfer agent can be omitted in the process.

Use of the acid salt of the arylamine as a starting point is optional in the route using water miscible solvent. The acid salt can be replaced with arylamine in the reaction scheme.

Stated more generally, when using the water miscible solvent route, the acid salt of arylamine is of the formula

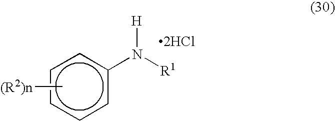

The other process conditions remain substantially similar when using the water miscible solvent. The different starting materials and solvents available to the synthetic chemist is reflective of the versatility of the invention for rapidly producing diphenylamines efficiently and in high yield by the process of the invention.

### EXAMPLE 1

### Preparation of 4-Methoxy-2-methyldiphenylamine

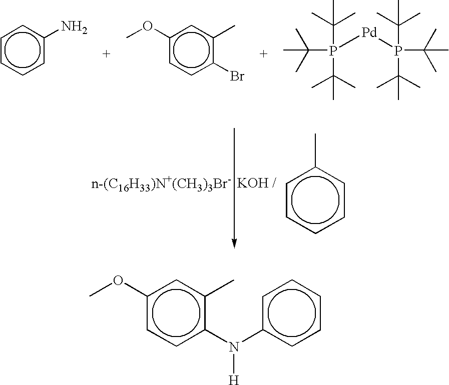

4-Bromo-3-methylanisole (10.0 g, 0.05 mole) and aniline (4.9 g, 0.05 mole) in toluene (50 ml) were placed in a 250 ml, three-necked, round-bottom flask equipped with a mechanical stirrer and a reflux condenser. Aqueous potassium hydroxide (5.0 g/10 ml of water) and cetyltrimethylammonium bromide (100 mg, 0.00027 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After 15 minutes, GC analysis of the reaction mixture showed that the reaction was complete. The reaction mixture was cooled to room temperature; diluted with water and brine stirred for few minutes; the toluene layer was separated and the aqueous layer was extracted twice with toluene. The toluene extracts were combined; washed with water, dried over anhydrous magnesium sulfate, filtered and the filtrate concentrated. The residue was distilled under vacuum. The product distilled over at 200-205° C./15 mm Hg. Yield 10.1 g (95%), Pale yellow liquid solidified on standing.

### EXAMPLE 2

### Preparation of 4-Methoxy-2,2′,4′-trimethyldiphenylamine

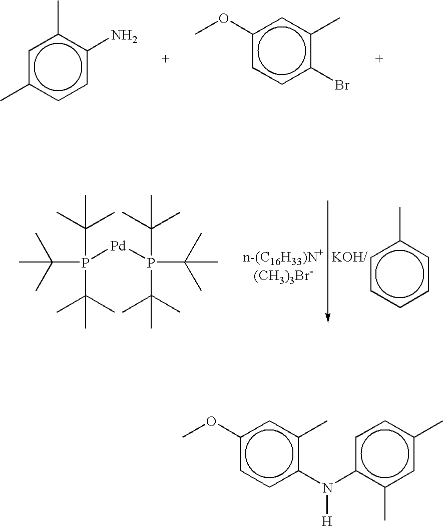

4-Bromo-3-methylanisole (10.0 g, 0.05 mole) and 2,4-dimethylaniline (6.1 g, 0.05 mole) in toluene (50 ml) were placed in a 250 ml, three-necked, round-bottom flask equipped with a mechanical stirrer and a reflux condenser. Aqueous potassium hydroxide (5.0 g/10 ml of water) and cetyltrimethylammonium bromide (100 mg, 0.00027 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After 15 minutes, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was distilled under vacuum. The product distilled over at 210-215° C./15 mm Hg. Yield 11.1 g (91%), Pale yellow liquid solidified on standing.

### EXAMPLE 3

### Preparation of N-Ethyl-3-methoxy-4′-methyldiphenylamine using 3-Bromoanisole

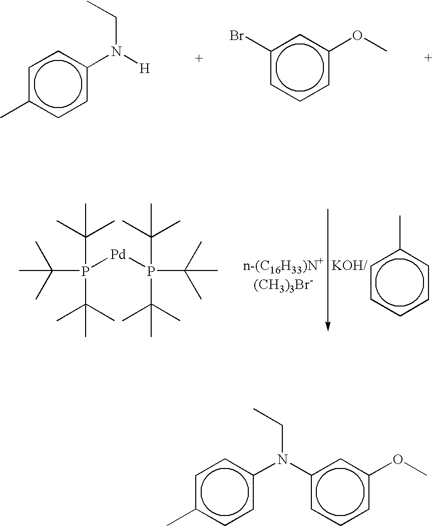

3-Bromoanisole (9.4 g, 0.05 mole) and N-methyl-4-toluidine (6.8 g, 0.05 mole) in toluene (50 ml) were placed in a 250 ml, three-necked, round-bottom flask equipped with a mechanical stirrer and a reflux condenser. Aqueous potassium hydroxide (5.0 g/10 ml of water) and cetyltrimethylammonium bromide (100 mg, 0.00027 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After 30 minutes, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was purified by column chromatography on silica gel using toluene as eluant. Fractions containing the product were collected, combined and concentrated under reduced pressure. Yield: 11.5 g (95%), Pale yellow liquid.

### EXAMPLE 4

### Preparation of N-Ethyl-3-methoxy-4′-methyldiphenylamine using 3-Chloroanisole instead of 3-Bromoanisole

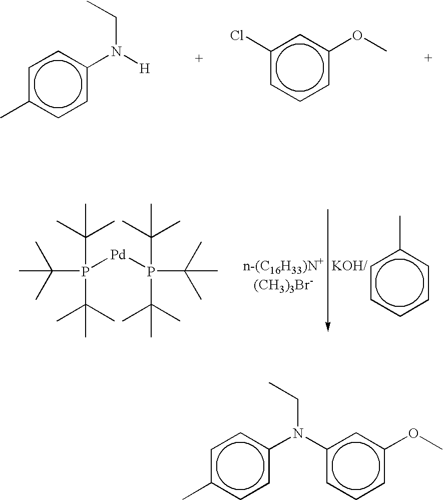

3-Chloroanisole (7.2 g, 0.05 mole) and N-ethyl-4-toluidine (6.3 g, 0.05 mole) in toluene (50 ml) were placed in a 250 ml, three-necked, round-bottom flask equipped with a mechanical stirrer and a reflux condenser. Aqueous potassium hydroxide (5.0 g/10 ml of water) and cetyltrimethylammonium bromide (100 mg, 0.00027 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After 2.5 hours, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was purified by column chromatography on silica gel using toluene as eluant. Fractions containing the product were collected, combined and concentrated under reduced pressure. Yield: 11.5 g (95%), Pale yellow liquid.

### EXAMPLE 5

### Preparation of 4-Dimethylamino-3′-methoxydiphenylamine using Sodium Hydroxide instead of Potassium Hydroxide

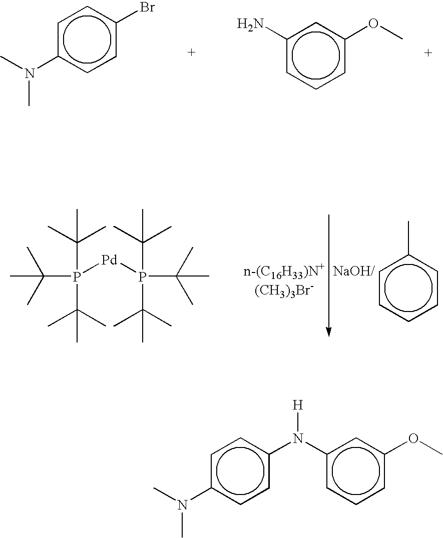

4-Bromo-N,N-dimethylaniline (10.0 g, 0.05 mole) and 3-anisidine (6.2 g, 0.05 mole) in toluene (50 ml) were placed in a 250 ml, three-necked, round-bottom flask equipped with a mechanical stirrer and a reflux condenser. Aqueous sodium hydroxide (5.0 g/10 ml of water) and cetyltrimethylammonium bromide (100 mg, 0.00027 mole) were added to the contents of the flask with stirring. After warming the flask to 90° C., bis[tri(t-butyl)phosphine]palladium (0) (250 mg, 0.0005 mole) was added and the progress of the reaction was monitored by gas chromatography (OV-1 column, 100° C. for 2 minutes, 25° C./minute to 300° C.). After one hour, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was recrystallized from methanol. Yield: 10.9 g (90%), Pale yellow solid, M.P.: 100-101° C.

### EXAMPLE 6

### Preparation of 4-Methoxy-2,2′,4′-trimethyldiphenylamine using Tetra-n-butylammonium Bromide as Phase-transfer agent

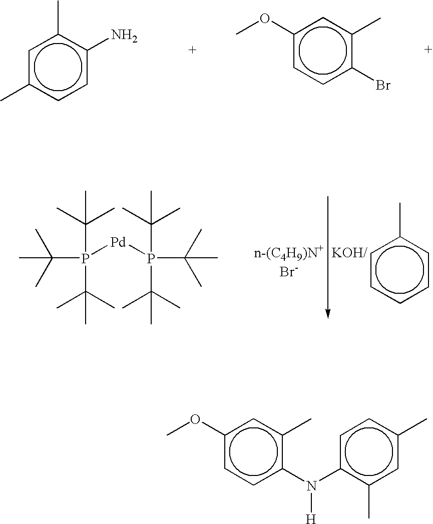

4-Bromo-3-methylanisole (10.0 g, 0.05 mole) and 2,4-dimethylaniline (6.1 g, 0.05 mole) in toluene (50 ml) were placed in a 250 ml, three-necked, round-bottom flask equipped with a mechanical stirrer and a reflux condenser. Aqueous potassium hydroxide (5.0 g/10 ml of water) and tetra-n-butylammonium bromide (100 mg, 0.0003 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After 2.5 hours, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was distilled under vacuum. The product distilled over at 210-215° C./15 mm Hg. Yield 10.8 g (90%), Pale yellow liquid solidified on standing.

### EXAMPLE 7

### Preparation of 4-Dimethylamino-3′-methoxydiphenylamine using N,N-Dimethyl-4-phenylenediamine dihydrochloride (in situ generation of aromatic amine)

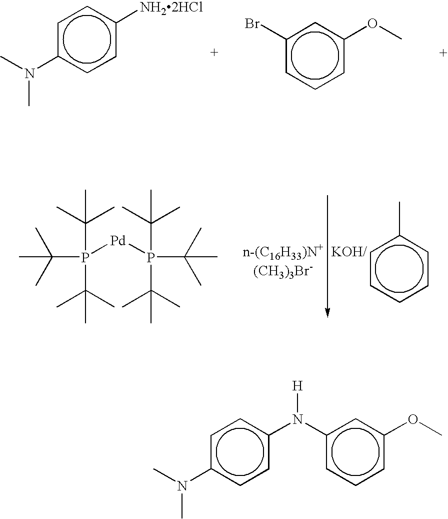

3-Bromoanisole (46.8 g, 0.25 mole) and N,N-dimethyl-4-phenylenediamine dihydrochloride (58.0 g, 0.28 mole) in toluene (300 ml) were placed in a one litre, three-necked, round-bottom flask equipped with a mechanical stirrer and a reflux condenser. Aqueous potassium hydroxide (60.0 g/120 ml of water) and cetyltrimethylammonium bromide (200 mg, 0.00054 mole) were added to the contents of the flask with stirring. After warming the flask to 90° C., bis[tri(t-butyl)phosphine]palladium [0] (250 mg, 0.0005 mole) was added and the progress of the reaction was monitored by gas chromatography (OV-1 column, 100° C. for 2 minutes, 25° C./minute to 300° C.). After one hour, GC analysis of the reaction mixture showed that the reaction was complete. The reaction mixture was cooled to room temperature; diluted with water and brine stirred for few minutes and the toluene layer was separated and the aqueous layer was extracted twice with toluene. The toluene extracts were combined; washed with water, dried over anhydrous magnesium sulfate, filtered and the filtrate concentrated. The residue was recrystallized from methanol. Yield: 53.2 g (88%), Pale yellow solid, M.P.: 100-101° C.

Unless otherwise indicated, all measurements herein are on the basis of weight and in the metric system.

The principles, preferred embodiments, and modes of operation of the present invention have been described in the following specification. The invention which is intended to be protected herein, however, is not to be construed as limited to the particular forms disclosed, since these are to be regarded as illustrative rather than restrictive. Variations and changes can be made by those skilled in the art without departing from the spirit and scope of the invention.

What is claimed is:

1. A novel process for the manufacture of diphenylamines of the formula 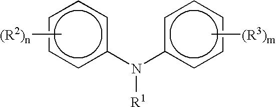
wherein R¹ is selected from hydrogen, alkyl, and aryl;
wherein each R² and R³ is the same or different and each is independently selected from hydrogen, alkyl, alkoxy, aralkyl, dialkylamino, alkylarylamino, pyrrollidino, piperidino, morpholino and substituted or unsubstituted aryl, the substituents on aryl being each independently selected from alkyl (C₁-C₈), alkoxy (C₁-C8), aroxy, aralkoxy and halogen;
wherein n and m are each independently an integer from 1 to 5;
the process comprising the steps of addition of an arylamine of the formula 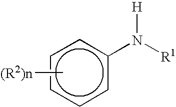
and an aryl halide of the formula 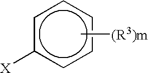
to a water immiscible solvent forming a mixture;
agitating the mixture;
adding an aqueous alkaline solution;
adding a phase transfer agent;
heating to a temperature of at least 40° C.;
and then next adding a catalytically effective amount of Pd[P(t-Bu)₃]₂.

2. The process according to claim 1 wherein the phase transfer agent is selected from cetyltrimethylammonium halide, tricaprylmethylammonium halide, tetra-n-butylammonium halide, quartemary ammonium salt, and quartemary phosphonium salt.

3. The process according to claim 1 wherein the water immiscible solvent is a hydrocarbon.

4. The process according to claim 1 wherein the solvent is toluene.

5. The process according to claim 1 wherein heating is to a temperature of at least 80° C.

6. The process according to claim 1 wherein the phase transfer agent is selected from cetyltrimethylammonium bromide, tricaprylmethylammonium chloride and tetra-n-butylammonium bromide.

7. A novel process for the manufacture of diphenylamines of the formula 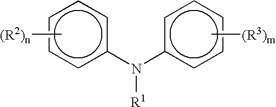
wherein R¹ is selected from hydrogen, alkyl, and aryl;
wherein each R² and R³ is the same or different and each is independently selected from hydrogen, alkyl, alkoxy, aralkyl, dialkylamino, alkylarylamino and substituted or unsubstituted aryl, the substituents on aryl being each independently selected from alkyl (C₁-C₈), alkoxy (C₁-C₈), aroxy, aralkoxy and halogen;
wherein n and m are each independently an integer from 1 to 5;
the process comprising the steps of addition of an acid salt of an arylamine, the arylamine having the formula 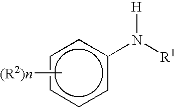
and an arylhalide of the formula 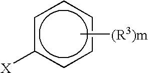
to a water immiscible solvent forming a mixture;
agitating the mixture;
adding an aqueous alkaline solution;
adding a phase transfer agent;
heating to a temperature of at least 40° C.;
and then adding a catalytically effective amount of Pd[P(t-Bu)₃]₂.

8. The process according to claim 7 wherein the phase transfer agent is selected from cetyltrimethylammonium halide, tricaprylmethylammonium halide, tetra-n-butylammonium halide, quartemary ammonium salt, and quartemary phosphonium salt.

9. The process according to claim 7 wherein the water immiscible solvent is a hydrocarbon.

10. The process according to claim 7 wherein the solvent is toluene.

11. The process according to claim 7 wherein heating is to a temperature of at least 80° C.

12. The process according to claim 7 wherein the phase transfer agent is selected from cetyltrimethylammonium bromide, tricaprylmethylammonium chloride and tetra-n-butylammonium bromide.

13. A novel process for the manufacture of diphenylamines of the formula 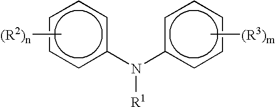
wherein R¹ is selected from hydrogen, alkyl, and aryl;
wherein each R² and R³ is the same or different and each is independently selected from hydrogen, alkyl, alkoxy, aralkyl, dialkylamino, alkylarylamino and substituted or unsubstituted aryl, the substituents on aryl being each independently selected from alkyl (C₁-C₈), alkoxy (C₁-C₈), aroxy, aralkoxy and halogen;
wherein n and m are each independently an integer from 1 to 5;
the process comprising the steps of addition of an acid salt of an arylamine, the arylamine having the formula 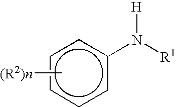
and an arylhalide of the formula 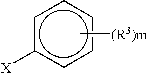
to a water miscible solvent forming a mixture;
adding an aqueous alkaline solution;
heating to a temperature of at least 40° C.;
and then next adding a catalytically effective amount of Pd[P(t-Bu)₃]₂.

14. The process according to claim 1 wherein the water miscible solvent is selected from 1,4-dioxane, diethylene glycol dimethylether, acetonitrile, dimethylsulfoxide, dimethylformamide, monopropylether, and ethylene glycol monopropyl ether.

15. The process according to claim 1 wherein heating is to a temperature of at least 80° C.

16. A novel process for the manufacture of diphenylamines of the formula 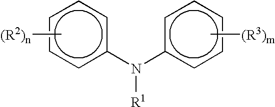
wherein R¹ is selected from hydrogen, alkyl, and aryl;
wherein each R² and R³ is the same or different and each is independently selected from hydrogen, alkyl, alkoxy, aralkyl, dialkylamino, alkylarylamino and substituted or unsubstituted aryl, the substituents on aryl being each independently selected from alkyl (C₁-C₈), alkoxy (C₁-C₈), aroxy, aralkoxy and halogen;
wherein n and m are each independently an integer from 1 to 5;
the process comprising the steps of addition of an arylamine of the formula 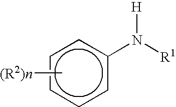
and an arylhalide of the formula 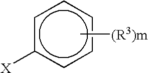
to a water miscible solvent forming a mixture;
adding an aqueous alkaline solution;
heating to a temperature of at least 40° C.; and then next adding a catalytically effective amount of Pd[P(t-Bu)₃]₂.

17. The process according to claim 1 including in addition phase transfer agent addition of a selected from cetyltrimethylammonium halide, tricaprylmethylammonium halide, tetra-n-butylammonium halide, quartemary ammonium salt, and quartemary phosphonium salt.

18. The process according to claim 16 wherein the water miscible solvent is selected from 1,4-dioxane, diethylene glycol dimethylether, acetonitrile, dimethylsulfoxide, dimethylformamide, monopropylether, and ethylene glycol monopropyl ether.

19. The process according to claim 16 wherein heating is to a temperature of at least 80° C.

20. The process according to claim 17 wherein the phase transfer agent is selected from cetyltrimethylammonium bromide, tricaprylmethylammonium chloride and tetra-n-butylammonium bromide.
